(19) United States
(12) Patent Application Publication
(10) Pub. No.: US 2016/0374703 A1
(43) Pub. Date: Dec. 29, 2016

(54) THROMBOLYSIS DEVICE AND METHOD OF OPERATING A THROMBOLYSIS DEVICE

(71) Applicant: AGENCY FOR SCIENCE, TECHNOLOGY AND RESEARCH, Connexis (SG)

(72) Inventors: Hongbin YU, Singapore (SG); Yuandong Alex GU, Singapore (SG); Liang LOU, Singapore (SG)

(73) Assignee: Agency for Science, Technology and Research, Singapore (SG)

(21) Appl. No.: 15/122,306

(22) PCT Filed: Mar. 20, 2015
(86) PCT No.: PCT/SG2015/000086

§ 371 Date: Aug. 29, 2016

(30) Foreign Application Priority Data
Mar. 20, 2014 (SG) 10201400821W

(51) Int. Cl. A61B 17/22; A61N 7/00; A61B 90/00; A61B 8/12

(52) U.S. Cl. CPC A61B 17/2202; A61B 8/12; A61N 7/00; A61B 90/37; A61N 2007/0043; A61B 2017/00084

(57) ABSTRACT

According to various embodiments, there is provided a thrombolysis device including a catheter configured to be inserted into a blood clot; a treatment transducer coupled to the catheter, the treatment transducer configured to transmit acoustic waves; a measurement transducer coupled to the catheter, the measurement transducer configured to transmit further acoustic waves and further configured to receive acoustic echoes, the acoustic echoes being reflections of the further acoustic waves from a boundary of the blood clot, wherein the measurement transducer is further configured to provide a measurement output; a determination circuit configured to determine at least one of a viscosity of blood in a vicinity of the measurement transducer or a volume of the blood clot, based on the measurement output; and a control circuit configured to generate control signals for controlling the treatment transducer, based on at least one from the group consisting of the viscosity of blood in the vicinity of the measurement transducer, the volume of the blood clot and a temperature measurement of the blood clot.

### CROSS-REFERENCE TO RELATED APPLICATIONS

This application claims benefit of Singapore Patent Application number 10201400821W filed 20 Mar. 2014, which is incorporated in its entirety herein by reference.

### TECHNICAL FIELD

The present invention relates to thrombolysis devices and methods of operating thrombolysis devices.

### BACKGROUND

The incidence of stroke is rising worldwide due to the increasing burden of chronic diseases such as hypertension and diabetes. For intracerebral hemorrhagic strokes, the clot in the brain can cause further peri-haematoma edema leading to a functional/neurological deterioration in patients. This neurological deterioration will lead to increased length of stay as well as a poorer outcome with increased disability. The resultant effect of this is an increased economic cost as length of stay has been found to be one of the largest costs in stroke management.

Medical treatment with blood pressure control only is the most conservative method without requiring any surgical operation, but patients on medical treatment alone will be at risk of neurological deterioration due to the pressure from the clot as well as increasing pen-haematoma edema as no attempt has been made on clot size reduction. In comparison, the most straightforward method is to remove clot by open surgery, however, clinical trials have shown no significant benefits with the surgical removal of small (<30 mls) and medium (30-60 mls) sized intracerebral hemorrhages. It has been theorized that this is due to the invasive nature of the surgery causing further neurological damage to normal brain tissue.

Minimally invasive surgical treatment with stereotactic aspiration or endoscopic aspiration with the possible addition of thrombolytic treatment and open surgical clot evacuation is another adopted method. In spite of the reduced brain damage risk, the variable clot removal rate at the time of surgery is a problem. At the same time, if these types of surgery are performed too early then there is a risk of causing a new bleeding event by rapid dissolution of the intracerebral clot, which is increased by the addition of thrombolytic agents as well as the addition of suction. These types of surgery are also resource intensive as they require the setup of endoscopic equipment in the operating theatre.

Ultrasound assisted sonothrombolysis has been validated in clot dissolution. One method is to use a low frequency transcranial focus ultrasound transducer for mechanical thrombolysis. Despite the efficacy and non-invasive characteristics, the expensive and bulky facility is a big concern. A minimally invasive method combining the ultrasound activation and recombinant tissue plasminogen activator (rt-PA) may be applied. In this case, an ultrasound catheter may be inserted into the brain for localized delivery of high frequency (2 MHz) ultrasound wave. It may facilitate the diffusion of rt-PA and increase its binding sites to fibrin so as to improve the clot lysis speed. However, the lack of control on the drug diffusion might induce bio-effect such as edema and rebleeding.

Therefore, there is a need for a new type of device for performing minimally invasive thrombolysis procedures without the need for thrombolysis drugs.

### SUMMARY

According to various embodiments, there may be provided a thrombolysis device, including a catheter, a treatment transducer, a measurement transducer, a determination circuit and a control circuit. The catheter may be configured to be inserted into a blood clot. The treatment transducer and the measurement transducer may be coupled to the catheter. The treatment transducer may be configured to transmit acoustic waves. The measurement transducer may be configured to transmit further acoustic waves and may be further configured to receive acoustic echoes, which are reflections of the further acoustic waves from a boundary of the blood clot. The measurement transducer may be further configured to provide a measurement output. The determination circuit may be configured to determine at least one of a viscosity of blood in a vicinity of the measurement transducer or a volume of the blood clot, based on the measurement output. The control circuit may be configured to generate control signals for controlling the treatment transducer, based on at least one from the group consisting of the viscosity of blood in the vicinity of the measurement transducer, the volume of the blood clot and a temperature measurement of the blood clot.

According to various embodiments, there may be provided a method of operating a thrombolysis device, the method including inserting a catheter into a blood clot; transmitting acoustic waves using a treatment transducer; transmitting further acoustic waves and receiving acoustic echoes using a measurement transducer; providing a measurement output using the measurement transducer; determining at least one of a viscosity of blood in a vicinity of the measurement transducer or a volume of the blood clot, based on the measurement output, using a determination circuit; and generating control signals for controlling the treatment transducer. The treatment transducer and the measurement transducer may be coupled to the catheter. The acoustic echoes may be reflections of the further acoustic waves from a boundary of the blood clot. The control signals may be generated based on at least one from the group consisting of the viscosity of blood in the vicinity of the measurement transducer, the volume of the blood clot and a temperature measurement of the blood clot.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the invention. In the following description, various embodiments are described with reference to the following drawings, in which:

FIG. 1 shows a conceptual diagram of a thrombolysis device in accordance to various embodiments.

FIG. 2 shows a conceptual diagram of a thrombolysis device in accordance to various embodiments.

FIG. 3 shows a flow diagram of a method of operating a thrombolysis device, in accordance to various embodiments.

FIG. 4 shows a schematic diagram of a thrombolysis device in operation, in accordance to various embodiments.

FIG. 5 shows an operation work flow of a thrombolysis device, in accordance to various embodiments.

FIG. 6 shows a cross-sectional view of a transducer element of a thrombolysis device, in accordance to various embodiments.

FIG. 7 shows a cross-sectional view of a transducer element of a thrombolysis device, in accordance to various embodiments.

FIGS. 8A to 8C show a transducer element of a thrombolysis device, in various stages of operation, according to various embodiments.

FIG. 9 shows a flow diagram for an acoustic pulse emitted by a thrombolysis device in accordance to various embodiments.

FIG. 10 shows a cross-sectional view of a blood clot with a catheter of a thrombolysis device inserted into the blood clot, in accordance to various embodiments.

FIG. 11 shows a finite element analysis model of a transducer element, in accordance to various embodiments.

FIG. 12 shows a simulated acoustic field distribution of the transducer element of FIG. 11.

FIG. 13 shows a graph of pressure against distance, for the simulated acoustic field distribution of FIG. 12.

FIG. 14 shows a simulated temperature field distribution within a blood clot when the transducer element of FIG. 11 is operated.

FIG. 15 shows a graph of temperature against distance, for the simulated temperature field distribution of FIG. 14.

FIG. 16 shows a schematic diagram of an experiment set-up for validating performance of a thrombolysis device, in accordance to various embodiments.

FIG. 17 shows a graph of output voltage against time, obtained from an experiment using the test set-up of FIG. 16.

FIG. 18 shows a graph of output voltage against time, as obtained from an experiment using the test set-up of FIG. 16.

FIG. 19 shows a graph of output voltage against time, as obtained from an experiment using the test set-up of FIG. 16.

FIG. 20 shows a graph of output voltage against time, as obtained from an experiment using the test set-up of FIG. 16.

FIG. 21 shows a graph of normalized amplitude of output voltage against frequency, as obtained from an experiment using the test set-up of FIG. 16.

### DESCRIPTION

Embodiments described below in context of the devices are analogously valid for the respective methods, and vice versa. Furthermore, it will be understood that the embodiments described below may be combined, for example, a part of one embodiment may be combined with a part of another embodiment.

In this context, the thrombolysis device as described in this description may include a memory which is for example used in the processing carried out in the thrombolysis device. A memory used in the embodiments may be a volatile memory, for example a DRAM (Dynamic Random Access Memory) or a non-volatile memory, for example a PROM (Programmable Read Only Memory), an EPROM (Erasable PROM), EEPROM (Electrically Erasable PROM), or a flash memory, e.g., a floating gate memory, a charge trapping memory, an MRAM (Magnetoresistive Random Access Memory) or a PCRAM (Phase Change Random Access Memory).

In an embodiment, a “circuit” may be understood as any kind of a logic implementing entity, which may be special purpose circuitry or a processor executing software stored in a memory, firmware, or any combination thereof. Thus, in an embodiment, a “circuit” may be a hard-wired logic circuit or a programmable logic circuit such as a programmable processor, e.g. a microprocessor (e.g. a Complex Instruction Set Computer (CISC) processor or a Reduced Instruction Set Computer (RISC) processor). A “circuit” may also be a processor executing software, e.g. any kind of computer program, e.g. a computer program using a virtual machine code such as e.g. Java. Any other kind of implementation of the respective functions which will be described in more detail below may also be understood as a “circuit” in accordance with an alternative embodiment.

In this context, the expression “acoustic waves” may refer to a type of longitudinal waves that propagate by means of adiabatic compression and decompression. Longitudinal waves are waves that have the same direction of vibration as their direction of travel. Acoustic waves travel with the speed of sound which depends on the medium they are passing through.

The incidence of stroke is rising worldwide due to the increasing burden of chronic diseases such as hypertension and diabetes. For intracerebral hemorrhagic strokes, the clot in the brain can cause further peri-haematoma edema leading to a functional/neurological deterioration in patients. This neurological deterioration will lead to increased length of stay as well as a poorer outcome with increased disability. The resultant effect of this is an increased economic cost as length of stay has been found to be one of the largest costs in stroke management.

Medical treatment with blood pressure control only is the most conservative method without requiring any surgical operation, but patients on medical treatment alone will be at risk of neurological deterioration due to the pressure from the clot as well as increasing peri-haematoma edema as no attempt has been made on clot size reduction. In comparison, the most straightforward method is to remove clot by open surgery, however, clinical trials have shown no significant benefits with the surgical removal of small (<30 mls) and medium (30-60 mls) sized intracerebral hemorrhages. It has been theorized that this is due to the invasive nature of the surgery causing further neurological damage to normal brain tissue.

Minimally invasive surgical treatment with stereotactic aspiration or endoscopic aspiration with the possible addition of thrombolytic treatment and open surgical clot evacuation is another adopted method. In spite of the reduced brain damage risk, the variable clot removal rates at the time of surgery are a problem. At the same time, if these types of surgery are performed too early then there is a risk of causing a new bleeding event by rapid dissolution of the intracerebral clot, which is increased by the addition of thrombolytic agents as well as the addition of suction. These types of surgery are also resource intensive as they require the setup of endoscopic equipment in the operating theatre.

Ultrasound assisted sonothrombolysis has been validated in clot dissolution. One method is to use a low frequency transcranial focus ultrasound transducer for mechanical thrombolysis. Despite the efficacy and non-invasive characteristics, the expensive and bulky facility is a big concern. A minimally invasive method combining the ultrasound activation and recombinant tissue plasminogen activator (rt-PA) may be applied. In this case, an ultrasound catheter may be inserted into the brain for localized delivery of high frequency (2 MHz) ultrasound wave. It may facilitate the diffusion of rt-PA and increase its binding sites to fibrin so as to improve the clot lysis speed. However, the lack of control on the drug diffusion might induce bio-effect such as edema and rebleeding.

Therefore, there is a need for a new type of device for performing minimally invasive thrombolysis procedures without the need for thrombolysis drugs.

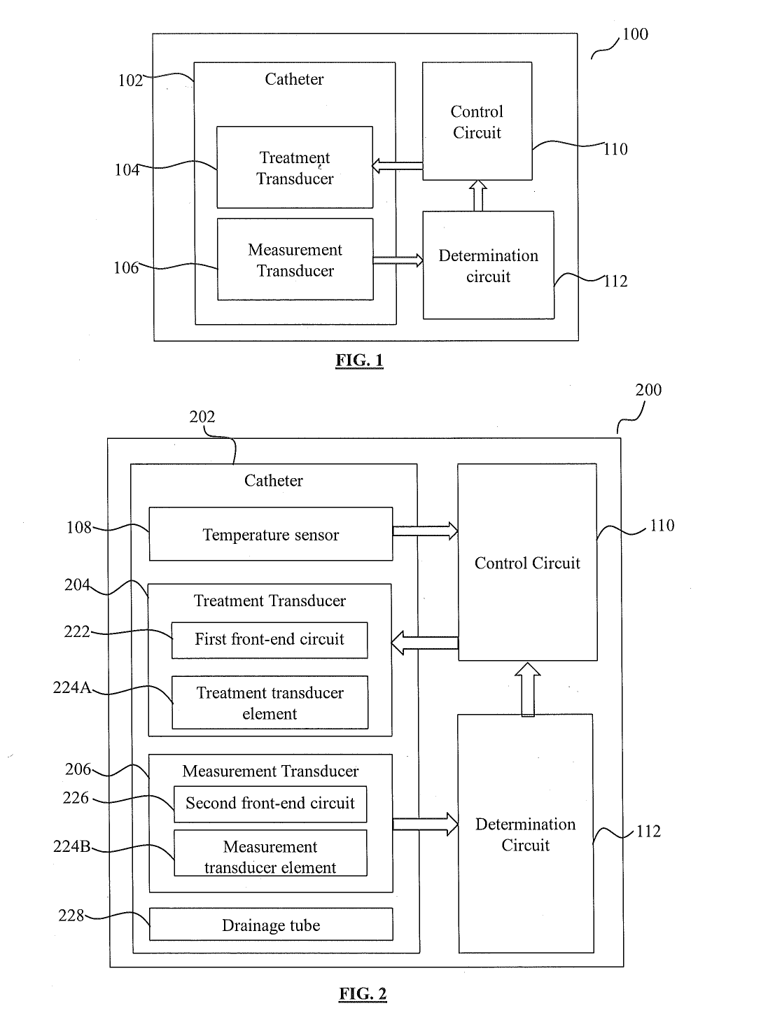

FIG. 1 shows a conceptual diagram of a thrombolysis device 100, in accordance to various embodiments. The thrombolysis device 100 includes a catheter 102, a treatment transducer 104, a measurement transducer 106, a control circuit 110 and a determination circuit 112. The catheter 102 may be configured to be inserted into a blood clot. The treatment transducer 104 which may be coupled to the catheter 102, may be configured to transmit acoustic waves. The measurement transducer 106 which may be coupled to the catheter 102, may be configured to transmit further acoustic waves and further configured to receive acoustic echoes. The acoustic echoes may be reflections of the further acoustic waves from a boundary of the blood clot. The measurement transducer 106 may be further configured to provide a measurement output. The determination circuit 112 may be configured to determine at least one of a viscosity of blood in a vicinity of the measurement transducer 106 or a volume of the blood clot, based on the measurement output from the measurement transducer 106. The control circuit 110 may be configured to generate control signals for controlling the treatment transducer 104, based on at least one of the viscosity of blood in the vicinity of the measurement transducer 106, the volume of the blood clot and a temperature measurement of the blood clot.

In other words, the thrombolysis device 100 includes a catheter 102, a treatment transducer 104, a measurement transducer 106, a determination circuit 112 and a control circuit 110. The catheter 102 may be configured for insertion into a blood clot, such as in a centre of the blood clot. The catheter 102 may be fabricated from a biocompatible and flexible material. The treatment transducer 104 and the measurement transducer 106 may be coupled to the catheter 102, for example, arranged on an end of the catheter 102 or on a sidewall of the catheter 102. The treatment transducer 104 and the measurement transducer 106 may be positioned within a tip of the catheter 102, wherein the tip is configured for insertion into the blood clot, so that the treatment transducer 104 and the measurement transducer 106 may come into contact with the blood clot when the catheter 102 is inserted into the blood clot.

The treatment transducer 104 may be configured to transmit acoustic waves. The acoustic waves may have a first frequency. The first frequency may be suitable for mechanically breaking down, or in other words, liquefying the blood clot. The acoustic waves transmitted by the treatment transducer 104 may be continuous acoustic waves. The measurement transducer 106 may be configured to transmit further acoustic waves and further configured to receive acoustic echoes. The acoustic echoes may be reflections of the further acoustic waves from a boundary of the blood clot. The further acoustic waves may be a series of acoustic pulses. The further acoustic waves and the acoustic echoes may have a second frequency, the second frequency being higher than the first frequency. The acoustic waves transmitted by the treatment transducer 104 and the further acoustic waves transmitted by the measurement transducer 106 may be ultrasound waves. The first frequency may lie within a range of 10 kHz to 100 kHz while the second frequency may lie within a range of 1 MHz to 10 MHz. The treatment transducer 104 may be structurally similar to the measurement transducer 106, but configured to transmit a different acoustic wave. The treatment transducer 104 and the measurement transducer 106 may also differ in physical size.

The measurement transducer 106 may be configured to provide a measurement output to the determination circuit 112. The measurement output may include at least one of a resonant frequency of the measurement transducer 106 and a travelling time of the acoustic echoes. The measurement output may further include information on an attenuation of the acoustic echoes, in other words, the difference in amplitude of the acoustic echoes as compared to the amplitude of the further acoustic waves transmitted by the measurement transducer 106. The determination circuit 112 may be configured to receive the measurement output for determining at least one of a viscosity of blood in a vicinity of the measurement transducer 106 or a volume of the blood clot. The determination circuit 112 may be further configured to determine the temperature measurement of the blood clot based on the measurement output.

The determination circuit 112 may determine the volume of the blood clot by computing a volume of a sphere having a radius, the radius being determined from the travelling time of the acoustic echoes. The determination circuit 112 may determine the viscosity of the blood in the vicinity of the measurement transducer 106 based on the resonant frequency of the measurement transducer 106. The determination circuit 112 may be further configured to provide at least one of the viscosity of blood in the vicinity of the measurement transducer 106 or the volume of the blood clot, to the control circuit 110. The determination circuit 112 may be further configured to determine the temperature measurement of the blood clot, based on an attenuation and delay of the further acoustic waves transmitted by the measurement transducer 106. The amount of attenuation of the further acoustic waves may depend on the temperature of the medium through which the further acoustic waves travel through. The delay in receiving the acoustic echoes may also depend on the temperature of the medium through which the further acoustic waves travel through. Therefore, the determination circuit 112 may determine the temperature of the blood clot, based on the measurement output which may contain information on at least one of the amount of attenuation of the further acoustic waves or the delay in receiving the acoustic echoes.

The control circuit 110 may be configured to generate control signals for controlling the treatment transducer 104, based on the information that it receives from the determination circuit 112. The control circuit 110 may be configured to generate the control signals based on at least one of the viscosity of blood in the vicinity of the measurement transducer, the volume of the blood clot and a temperature measurement of the blood clot. The temperature measurement of the blood clot may be provided by a temperature sensor integrated into the catheter. The temperature measurement of the blood clot may also be provided by the determination circuit 112, which may determine the temperature measurement of the blood clot based on the measurement output from the measurement transducer 106.

FIG. 2 shows a conceptual diagram of a thrombolysis device 200, in accordance to various embodiments. Similar to the thrombolysis device 100, the thrombolysis device 200 includes a catheter 202, a treatment transducer 204, a measurement transducer 206, a determination circuit 112 and a control circuit 110. The catheter 202 may be inserted into a blood clot. The treatment transducer 204 and the measurement transducer 206 may be coupled to the catheter 202. The treatment transducer 204 may be configured to transmit acoustic waves. The measurement transducer 206 may be configured to transmit further acoustic waves and may be further configured to receive acoustic echoes which are the reflections of the further acoustic waves from a boundary of the blood clot. The measurement transducer 206 may be further configured to provide a measurement output. The determination circuit 112 may be configured to determine at least one of a viscosity of blood in a vicinity of the measurement transducer 206 or a volume of the blood clot, based on the measurement output. The control circuit 110 may be configured to generate control signals for controlling the treatment transducer 204, based on at least one of the viscosity of the blood in the vicinity of the measurement transducer 206, the volume of the blood clot and a temperature measurement of the blood clot.

In comparison to the thrombolysis device 100 of FIG. 1, the thrombolysis device 200 further includes a temperature sensor 108, a drainage tube 228, a first front-end circuit 222, at least one treatment transducer element 224A, a second front-end circuit 226 and at least one measurement transducer element 224B. The temperature sensor 108 may be configured to provide the temperature measurement of the blood clot. The temperature sensor 108 may be coupled to the catheter 202 or positioned within the catheter 202. The temperature sensor 108 may be arranged on an end of the catheter 202 or on a sidewall of the catheter 202. The temperature sensor 108 may be positioned within a tip of the catheter 202, wherein the tip is configured for insertion into the blood clot, so that the temperature sensor 108 may come into contact with the blood clot when the catheter 202 is inserted into the blood clot.

The drainage tube 228 may be coupled to the catheter 202 and may be configured to drain blood out of the blood clot. The treatment transducer 204 may include the first front-end circuit 222 and the at least one treatment transducer element 224A. The measurement transducer 206 may include the second front-end circuit 226 and the at least one measurement transducer element 224B. The first front-end circuit 222 and the second front-end circuit 226 may be configured to control, in other words, drive the treatment transducer elements 224A and the measurement transducer elements 224B, respectively. The treatment transducer elements 224A and the measurement transducer elements 224B may be miniaturized, micromachined ultrasound transducers designed and fabricated based on MEMS technology. The measurement transducer elements 224B may be similar to the treatment transducer elements 224A in structure, but may have differing dimensions from the treatment transducer elements 224A. The treatment transducer elements 224A and the measurement transducer elements 224B may be fabricated on a common semiconductor device, as an array having one or two dimensions. Each of the treatment transducer elements 224A and each of the measurement transducer elements 224B may include a suspended membrane with a clamped boundary. When operating under a transmission mode, the suspended membrane may be driven to vibrate so as to emit an acoustic wave into the environment. In a receiving mode, an incoming acoustic wave may force the membrane to vibrate. Various driving methods, such as electrostatic and piezoelectric, may be used, with respect to which, the structure of the treatment transducer elements 224A and the measurement transducer elements 224B as well as its requirement on the first front-end circuit 222 and the second front-end circuit 224 respectively, may also be different.

The treatment transducer element 224A may emit an acoustic wave having a first frequency for liquefying the blood clot. The measurement transducer element 224B may operate at a resonant frequency which varies with a viscosity of the blood that the measurement transducer element 224B comes into contact with. The blood viscosity value can therefore be determined from the resonant frequency of the measurement transducer element 224B. When the measurement transducer element 224B vibrates, an acoustic pulse is transmitted. The acoustic pulse is reflected off a boundary of the blood clot and returns to the measurement transducer element 224B as an acoustic echo. The time taken for the acoustic echo to reach back to the measurement transducer element 224B depends on a radius of the blood clot. By assuming the blood clot is of a spherical shape, the volume of the blood clot may be approximated based on the travelling time of the acoustic echo.

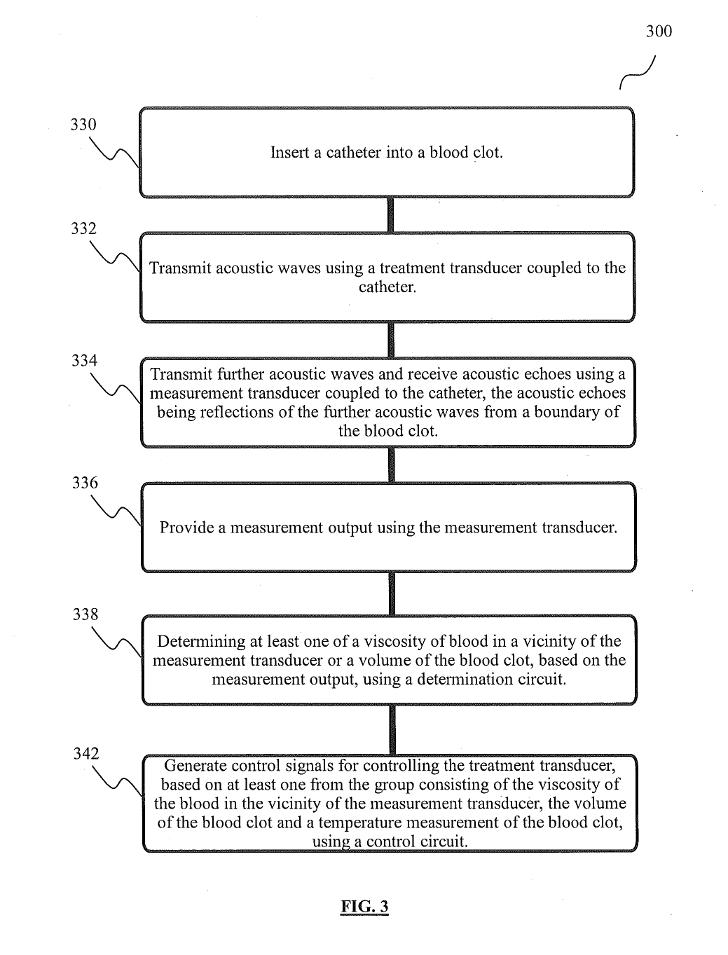

FIG. 3 shows a flow diagram 300 showing a method of operating a thrombolysis device, in accordance to various embodiments. In 330, a catheter may be inserted into a blood clot. In 332, acoustic waves may be transmitted using a treatment transducer which is coupled to the catheter. In 334, further acoustic waves may be transmitted and acoustic echoes may be received, using a measurement transducer coupled to the catheter. The acoustic echoes may be reflections of the further acoustic waves from a boundary of the blood clot. In 336, a measurement output may be provided, using the measurement transducer. In 338, at least one of a viscosity of blood in a vicinity of the measurement transducer or a volume of the blood clot may be determined based on the measurement output, using a determination circuit. In 342, control signals may be generated for controlling the treatment transducer, based on at least one of the viscosity of blood in the vicinity of the measurement transducer, the volume of the blood clot and a temperature measurement of the blood clot. The method 300 may further include using neuronavigation techniques to insert the catheter into a centre of the blood clot.

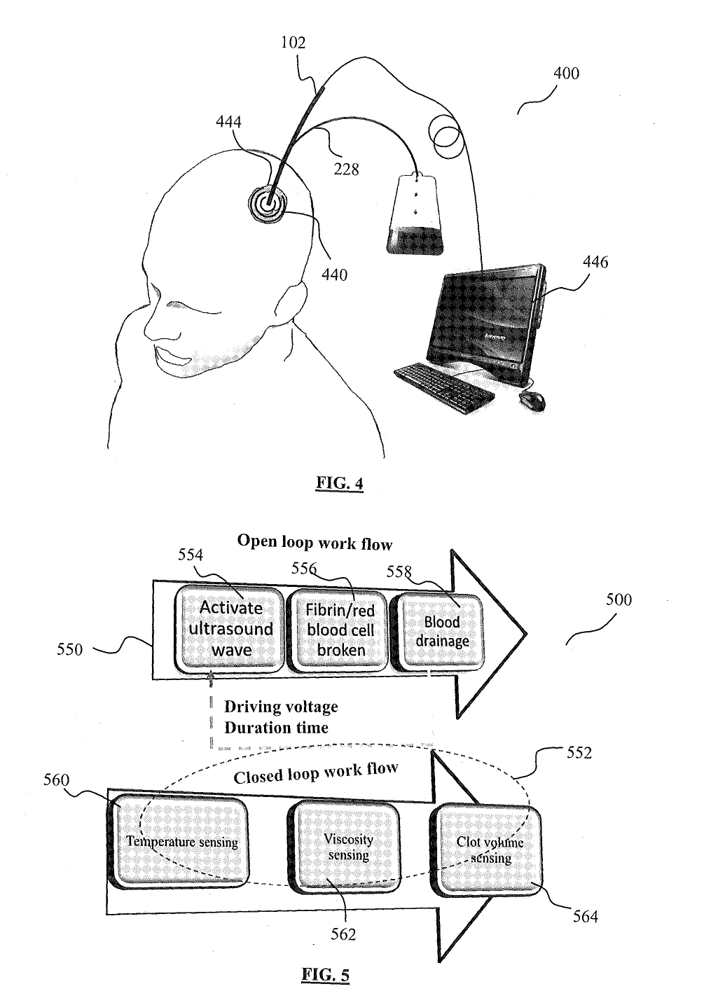

FIG. 4 shows a schematic diagram 400 of a thrombolysis device in operation, in accordance to various embodiments. The thrombolysis device may be an ultrasound catheter based, minimally invasive sonothrombolysis device for treating hemorrhagic strokes. The thrombolysis device may function by using ultrasound wave alone to accelerate lysis rate of a blood clot, thereby lowering the risk of neurological deterioration due to edema or mechanical pressure by an intracerebral blood clot. To operate the thrombolysis device, a catheter 102 may first be inserted into a blood clot 444 for a localized delivery of acoustic waves 440 to the blood clot 444. The catheter 102 may be inserted accurately into a centre of the blood clot 444, using standard neuronavigation techniques. The catheter 102 may include a treatment transducer and a measurement transducer. The treatment transducer and the measurement transducer may each include a plurality of transducer elements operating under low frequencies and high frequencies, respectively. The low frequencies may be in the range of tens of kilohertz while the high frequencies may be in the range of several megahertz. When the low frequency transducer elements in the treatment transducer are activated, the blood clot may be mechanically broken down by the low frequency ultrasound waves alone. The dissolved blood clot may be drained out by gravity, through a drainage tube 228 without any additional suction force. When the high frequency transducer elements are activated, they may transmit high frequency acoustic pulses to perform in-situ blood clot viscosity sensing and approximation of the blood clot volume. At the same time, a local temperature rise during treatment may be monitored with a temperature sensor integrated into the catheter 102. Information on the blood clot viscosity, the blood clot volume and the local temperature rise may be used to validate the treatment efficacy and provide real-time feedback information to a closed-loop control circuit. The control circuit may be housed within a control terminal 446, such as a computer system. The control circuit may provide control signals for controlling operation parameters of the treatment transducer, such as a driving voltage and a duration time of operation.

FIG. 5 shows an operation work flow 500 of a thrombolysis device, in accordance to various embodiments. The operation work flow 500 may include an open loop work flow 550 and a closed loop work flow 552. The open loop work flow 550 may include 554 where the transmission of ultrasound waves is activated; 556 where fibrin or red blood cell of a blood clot is broken resulting in liquefied blood and; 558 where the liquefied blood is drained away. The closed loop work flow may include 560 where the thrombolysis device senses a temperature of the blood clot; 562 where the thrombolysis device senses a viscosity of blood and 564 where the thrombolysis device senses the blood clot volume. The closed loop work flow may further include controlling the transmission of ultrasound waves through parameters such as a driving voltage and a transmission duration, based on information provided by 560, 562 and 564.

FIG. 6 shows a Capacitive Micromachined Ultrasonic Transducer (CMUT) 600, in accordance to various embodiments. The CMUT 600 may be used as at least one of the treatment transducer element 224A or the measurement transducer element 224B of FIG. 2. The CMUT 600 may include a CMUT membrane 660, an insulation layer 664, a top electrode 666 and a bottom electrode 668. The CMUT membrane 660 may be suspended over a cavity 662, wherein at least one end of the CMUT membrane 660 is anchored to the insulation layer 664. The cavity 662 may be formed within the insulation layer 664. The top electrode 666 may be positioned over a top surface of the CMUT membrane 660, wherein the top surface of the CMUT membrane 660 opposes a bottom surface of the CMUT membrane 660. The bottom surface of the CMUT membrane 660 may face the cavity 662. The bottom electrode 668 may be positioned under the insulator layer 664. The bottom electrode 668 may be in contact with a bottom surface of the insulator layer 664. The bottom surface of the insulator layer 664 opposes a top surface of the insulator layer 664, wherein the top surface of the insulator layer 664 is in contact with the cavity 662. The membrane 660 may be fabricated from silicon.

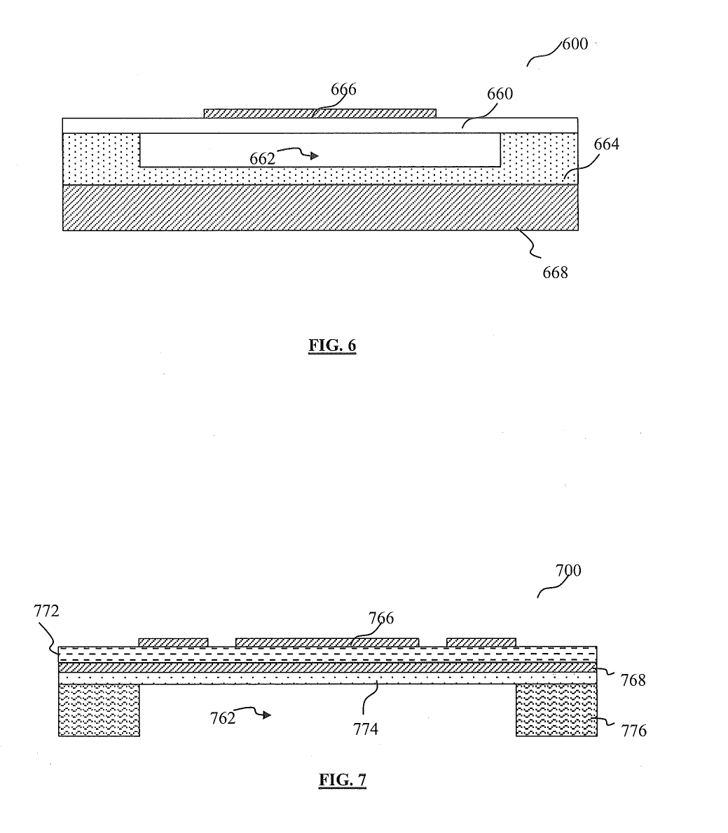

FIG. 7 shows a Piezoelectric Micromachined Ultrasonic Transducers (pMUT) 700, according to various embodiments. The pMUT 700 may be used as at least one of the treatment transducer element 224A or the measurement transducer element 224B of FIG. 2. The pMUT 700 may include a piezoelectric layer 772, a top electrode 766, a bottom electrode 768, a support layer 774 and a substrate 776. The piezoelectric layer 772 may be sandwiched between the top electrode 766 and the bottom electrode 768. The top electrode 766 may be patterned such that it only partially overlaps the piezoelectric layer 772. The bottom electrode 768 may be positioned in contact with the support layer 774, such that the bottom electrode 768 has a first surface in contact with the piezoelectric layer 772 and a second surface in contact with the support layer 774, wherein the second surface opposes the first surface. Collectively, the top electrode 766, the piezoelectric layer 772, the bottom electrode 768 and the support layer 774 form a pMUT membrane, which may be suspended over a cavity 762. The pMUT membrane may have both ends fixed to the substrate 776. The pMUT membrane may also be fixed to the substrate 776 at only one end.

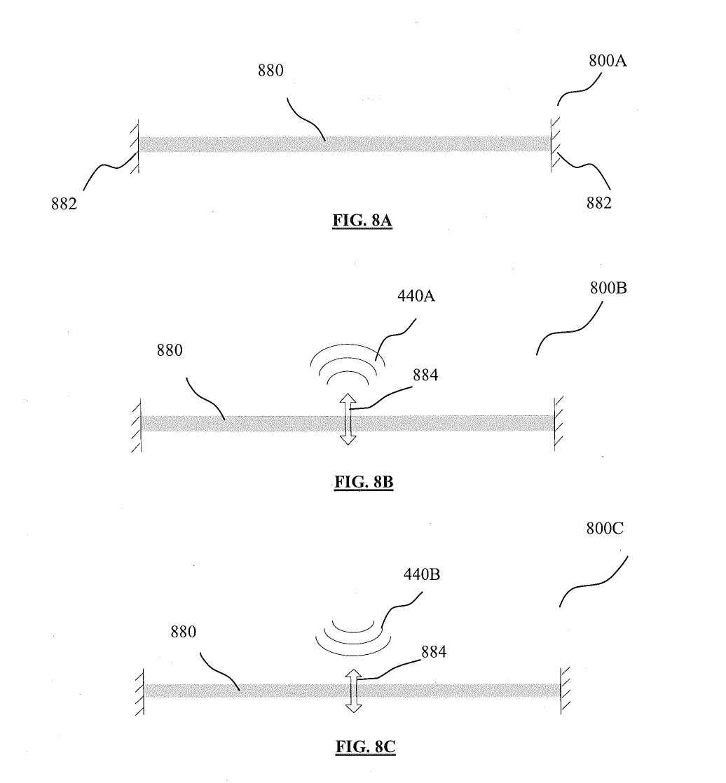

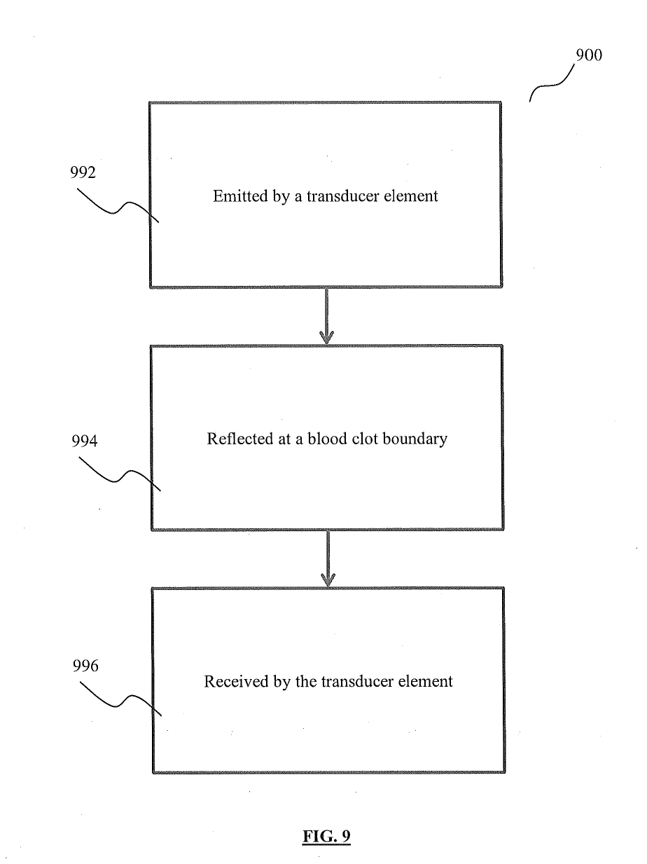

FIGS. 8A to 8C shows a transducer element in various stages of operation, according to various embodiments. The transducer element may be a treatment transducer element or a measurement transducer element. The transducer element may be the CMUT 600 of FIG. 6 or the pMUT 700 of FIG. 7. The transducer element may also be other types of micromachined transducers.

FIG. 8A shows the transducer element in a stationary mode 800A. The transducer element includes a membrane 880 with at least one end affixed to an anchor 882. The membrane 880 may also be affixed to anchors 882 at more than one end. FIG. 8B shows the transducer element in a transmitting mode 800B. In the transmitting mode 800B, the transducer element may be operated in a flexural mode, in other words, the membrane 880 may be actuated to vibrate in a transverse direction 884, the transverse direction being perpendicular to a plane of the membrane 880. The vibrations from the membrane 880 result in an outgoing acoustic wave 440A being emitted. FIG. 8C shows the transducer element in a receiving mode 800C. In the receiving mode 800C, the membrane 880 may vibrate as a result of incoming acoustic waves 440B. The vibrations of the membrane 880 may be converted into electrical signals.

For transducer elements operating in the flexural mode, the operating frequency may be determined solely by the mechanical property of the suspended membrane regardless of the driving method. The fundamental resonant frequency f of a membrane with clamped boundary condition in vacuum may be described by Equation (1), as follows:

$$f = \{ \begin{matrix}\frac{40.8 \cdot h}{8 π \cdot {r}^{2}} \sqrt{\frac{E}{12 \cdot ρ \cdot ( 1 - {v}^{2} )}} & ( circular membrane ) \\ \frac{35.16 \cdot h}{8 π \cdot a \cdot b} \sqrt{\frac{E}{12 \cdot ρ \cdot ( 1 - {v}^{2} )}} & ( square or rectangular membrane )\end{matrix} \qquad ( 1 )$$

where E, v and ρ are the Young's modulus, the Poisson's ratio and the density of the membrane material, respectively. r, a and b are the membrane radius, membrane width and membrane length, respectively while h is the membrane thickness. As can be seen from Equation (1), for any h value, the fundamental resonant frequency f may be varied by changing r in the case of a circular membrane or at least one of a or b, for a square or rectangular membrane. Therefore, a plurality of ultrasound transducers configured for operating at different frequencies may be simultaneously fabricated on a same substrate by designing differing lateral membrane dimensions for each ultrasound transducer within the plurality of ultrasound transducers.

Besides the operating frequency, the acoustic intensity is another key parameter that can directly determine the efficacy of the acoustic wave based sonothrombolysis. For ultrasound devices, the acoustic intensity W is proportional to the square of the generated acoustic pressure P, as follows:

$$W = \frac{{P}^{2}}{ρ c}$$

where ρ is the density of the acoustic wave transmission medium and c is the acoustic wave transmission speed.

For ultrasound transducers working under the flexural mode, the acoustic pressure generation capability is dependent on the membrane deflection frequency (f) and average amplitude (dₘ), as follows:

P=\sqrt{2}πf·d_{avg}·ρc

where d_{avg} is governed by the deflection function

$$D [ \frac{{\partial }^{4} d}{\partial {x}^{4}} + 2 \frac{{\partial }^{4} d}{\partial {x}^{2} \partial {y}^{2}} + \frac{{\partial }^{4} d}{\partial {y}^{4}} ] = p ( x , y )$$

where p is the driving force and D is the flexural rigidity of the membrane

$$D = \frac{E \cdot {h}^{3}}{12 ( 1 - {v}^{2} )}$$

During operation of the thrombolysis device, the ultrasound transducer may be in contact with the blood as the catheter housing the ultrasound transducer is inserted into the blood clot. The vibrating energy generated by the ultrasound transducer may be partially dissipated by acoustic radiation and viscous damping, the amount of which will be dependent on the viscosity of the blood clot surrounding the ultrasound transducer. Therefore, the resonant frequency of the ultrasound transducer in contact with blood, f_{blood}, will be changed in accordance to Equation (2).

$${f}_{blood} = \frac{f}{\sqrt{1 + β}} \qquad ( 2 )$$

where β is the added virtual mass by blood clot.

With respect to the dynamic viscosity η of fluid, β can be expressed by

$$β = \{ \begin{matrix}0.669 \frac{{ρ}_{blood} \cdot r}{ρ \cdot h} & ( {Lamb}^{′} s model , for η \leq 10 cP ) \\ \begin{matrix}0.6538 \frac{{ρ}_{blood} \cdot r}{ρ \cdot h} \cdot \\ ( 1 + 1.082 ξ )\end{matrix} & ( {Kozlovsky}^{′} s model , for η > 10 cP ) ( 3.2 )\end{matrix} \qquad ( 3.1 )$$

where ρ_{blood} is the density of the blood clot, ξ is a parameter characterizing the energy dissipation, r is the membrane radius and h is the membrane thickness.

$$ξ = \sqrt{\frac{η}{{ρ}_{blood} \cdot 2 π {f}_{blood} \cdot {r}^{2}}} \qquad ( 4 )$$

An ultrasound transducer, in accordance to various embodiments, may for example, operate at a fundamental resonant frequency of 27.1 MHz when the ultrasound transducer is in contact with normal blood having a dynamic viscosity, η=3.5 cP and density, ρ_{blood}=1.08×10³ kg/m³, the resonant frequency falls to 15.9 MHz, as may be determined from Equation (2) and (3.1). Assuming the membrane supporting layer is made of silicon, the membrane radius may be within the range of 10 μm to 40 μm, while the membrane thickness may be within the range of 1 μm to 10 μm.

If the ultrasound transducer is surrounded by a blood clot, under the effect of higher viscosity (η15 cP), the resonant frequency will be further reduced to 12.18 MHz using Equations (2), (3.2) and (4). For the purpose of the calculations, the membrane radius (r) is 27 μm, the membrane thickness (h) is 5 μm and the value of density of membrane material (ρ) is assumed to be 2330 kg/m³, which is the density of silicon.

Therefore, the viscosity variation of the blood clot during the treatment may be monitored in real time by measuring the frequency response of the ultrasound device with a pulse-echo operation.

FIG. 9 shows a flow diagram 900 for an ultrasound pulse emitted by a thrombolysis device, in accordance to various embodiments. In 992, the ultrasound pulse is emitted by a transducer element. The ultrasound pulse travels towards a boundary of the blood clot. In 994, the ultrasound pulse is reflected at the boundary and travels back to the transducer element. In 996, the reflected ultrasound pulse is received by the transducer element.

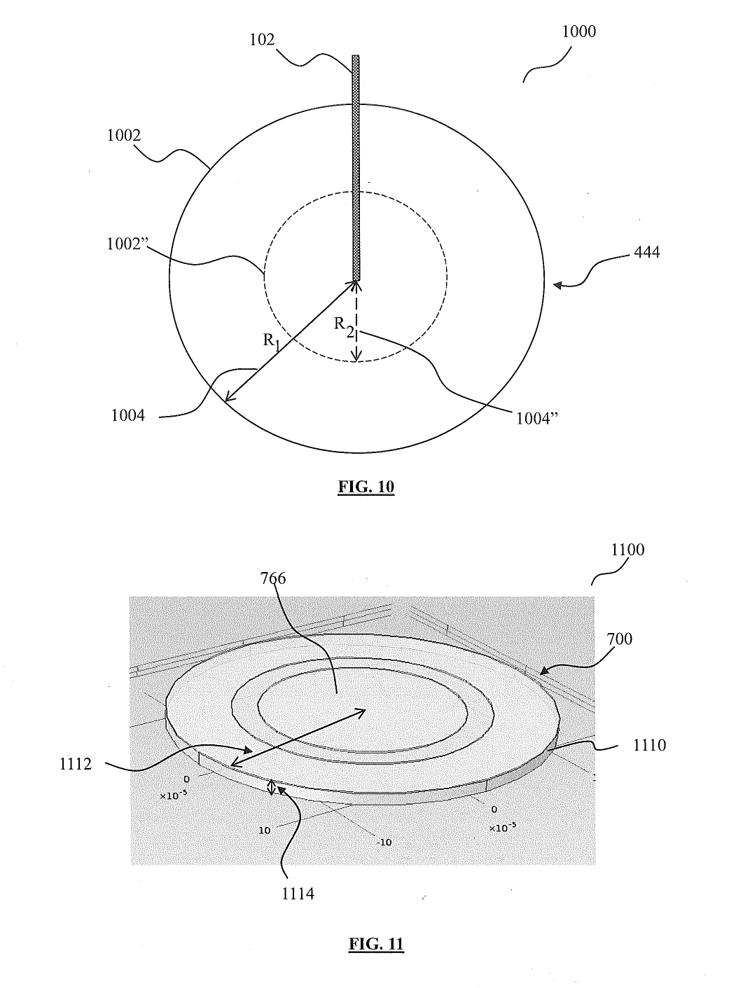

FIG. 10 shows a cross-sectional diagram 1000 of a blood clot 444 being measured by a thrombolysis device, in accordance to various embodiments. A catheter 102 may be inserted into a centre of the blood clot 444. The shape of the blood clot 444 may be approximated as a sphere. The blood clot 444 may have a first boundary 1002, prior to operation of the thrombolysis device. A first acoustic pulse may be emitted from a tip of the catheter 102, the tip being at the centre of the blood clot 444. The first acoustic pulse may travel through a first distance 1004 (R₁), to reach the first boundary 1002 and may be reflected off the first boundary 1002. The reflected first acoustic pulse travels back to the catheter 102 through the first distance 1004. The time taken for the first acoustic pulse to travel to the first boundary 1002 and back to the catheter 102 may be represented as 2t₁. At a next time instance, the blood clot may have shrunken in size due to acoustic waves emitted by the treatment transducer, such that the blood clot boundary shifts inwards to a second boundary 1002″. At the next time instance; the catheter 102 may emits a second acoustic pulse, which travels through a second distance 1004″ (R₂), to reach the second boundary 1002″ and may be reflected off the second boundary 1002″. The reflected second acoustic pulse then travels back to the catheter through the second distance 1004″. The time taken for the second acoustic pulse to travel to the second boundary 1002″ and back to the catheter 102 may be represented as 2t₂.

The first distance R₁ and the second distance R₂ may be determined from Equation (5):

R₁=t₁·c,R₂=t₂·c (5)

where c is the propagation speed of the acoustic pulse.

Approximating the blood clot to be spherical, the reduction in the blood clot volume, ΔV may be determined from Equation (6):

$$Δ V = {V}_{1} - {V}_{2} = \frac{4 π}{3} ( {R}_{1}^{3} - {R}_{2}^{3} ) \qquad ( 6 )$$

where V₁ is the volume of the blood clot having the first boundary 1002 and V₂ is the volume of the blood clot having the second boundary 1002″. The blood volume reduction ΔV may be used as a measure for evaluating the sonothrombolysis efficacy.

In the following, a Finite Element Analysis (FEA) of a treatment transducer, according to various embodiments, will be described. The FEA is performed to validate the technical feasibility, and safety of the treatment transducer in treating a blood clot.

FIG. 11 shows a FEA model 1100 of a treatment transducer, according to various embodiments. The treatment transducer may be a pMUT device 700. The pMUT device 700 may include a pMUT membrane 1110 suspended over a cavity formed within a substrate. The pMUT membrane 1110 may include a top electrode 766 and a bottom electrode 768 (not shown). The pMUT membrane 1110 may be circular in shape and may have a radius 1112 and a thickness 1114 of 350 μm and 5 μm, respectively. The fundamental resonant frequency of the pMUT device 700 may be 35 kHz, which falls into the effective frequency range for sonothrombolysis treatment.

FIG. 12 shows a simulated acoustic field distribution 1200 of the FEA model 1100 of FIG. 11. The simulated acoustic field distribution 1200 shows that an acoustic pressure is at its maximum value 1220 at a centre of the pMUT device and attenuates to a minimum value 1222 as the acoustic wave travels away from the centre of the pMUT device.

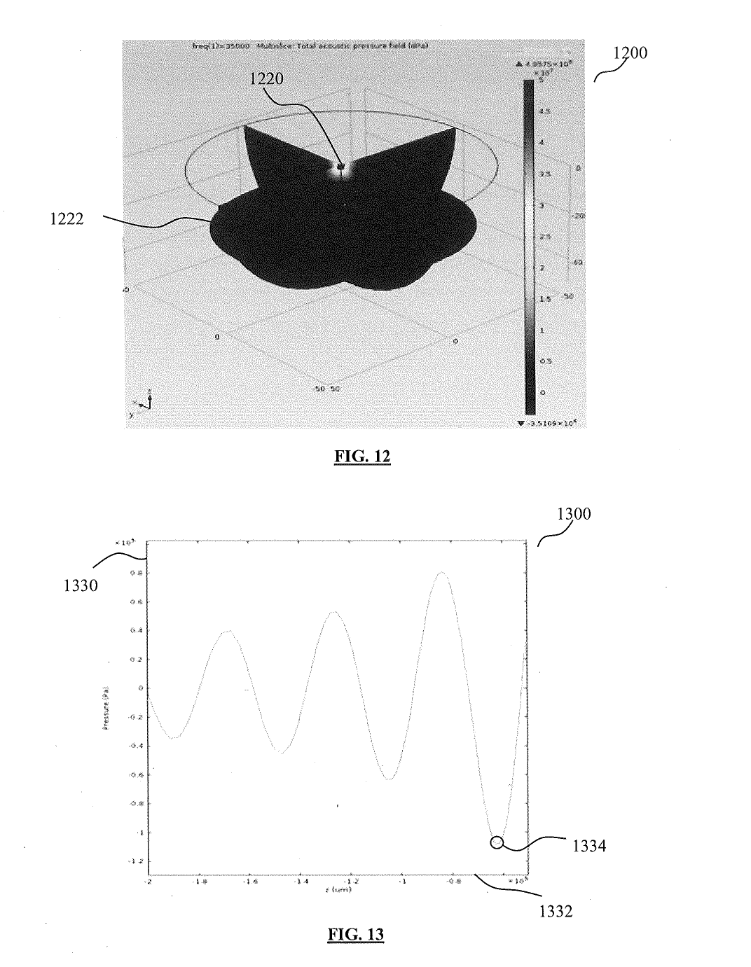

FIG. 13 shows a graph 1300 corresponding to the simulated acoustic field distribution 1200 of FIG. 12. The graph 1300 has a vertical axis 1330 indicating the acoustic pressure and a horizontal axis 1332 indicating the distance away from the centre of the pMUT device. A point 1334 on the graph 1300 shows that the acoustic pressure is around 110 kPa, 6 cm away from the centre of the pMUT device. As the targeted blood clot volume to be treated is about 30 ml to 50 ml, and the clot is usually treated as a spherical shape, the equivalent radius of the blood clot is calculated to be about 2.3 cm. From published data, an acoustic pressure of around 85 kPa is high enough for effective treatment. As such, the simulated acoustic field distribution 1200 validates the feasibility of using the pMUT device as a treatment transducer for mechanically breaking down a blood clot.

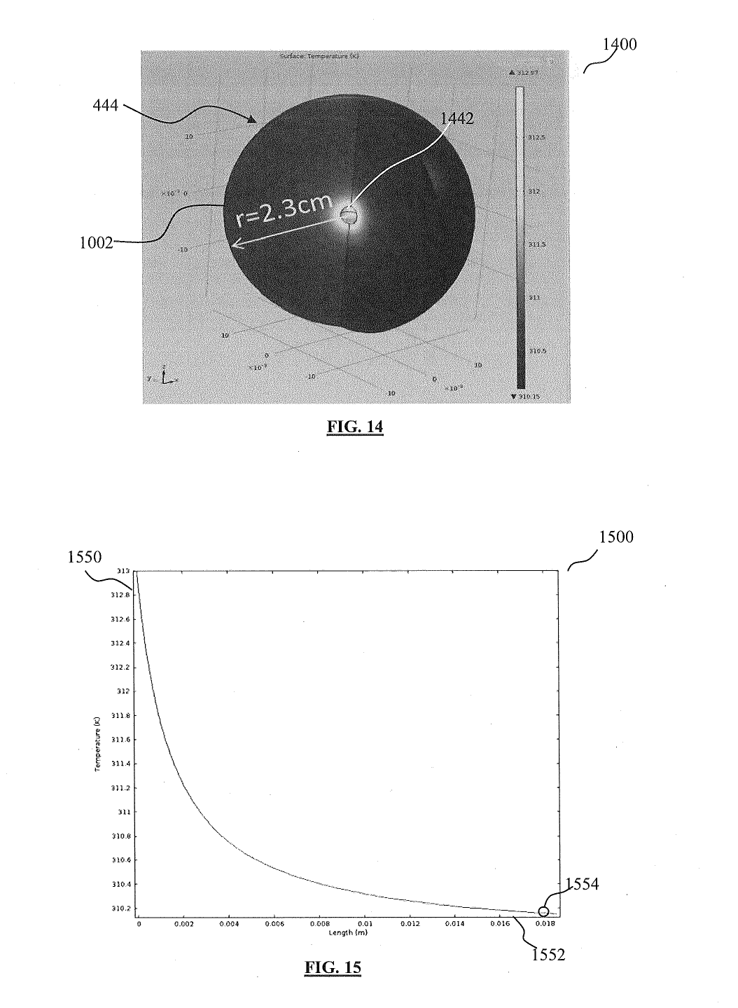

FIG. 14 shows a simulated temperature field distribution 1400 within a blood clot 444, when the blood clot 444 is being treated by a treatment transducer, in accordance to various embodiments. A thermal simulation was performed, to estimate the safety of the treatment transducer in accordance to various embodiments. Besides efficacy, safety is also an important consideration for a sonothrombolysis device, as at least part of the acoustic energy from the acoustic waves emitted by the treatment transducer may be converted to thermal energy. The upper safety temperature limit for brain tissues is typically about 41° C. to 42° C., above which the brain tissues will suffer from negative bio-effects. For a more conservative assessment of the safety of the treatment, a centre 1442 of the blood clot 444 was simulated as having reached 43° C., higher than the upper safety temperature limit. During the simulation, the values of specific heat and thermal conductivity for human clotted blood of 3.5×10³ J/kg·K and 0.59 W/m·K, respectively, were adopted. The blood clot 444 was modeled as having a radius of 2.3 cm. The temperature at a boundary 1002 of the blood clot was measured, to assess the safety of the treatment. The simulated temperature field distribution 1400 shows that the majority of the blood clot 444 has a lower temperature than the centre 1442 of the blood clot 444.

FIG. 15 shows a graph 1500 corresponding to the simulated temperature field distribution 1400 of FIG. 14. The graph 1500 has a vertical axis 1550 indicating the temperature measured in Kelvins and a horizontal axis 1552 indicating the distance away from the centre of the blood clot. A point 1554 on the graph 1500 shows that the temperature of the blood clot, at a distance of 1.8 cm away from the centre of the blood clot, falls to about 310.1K (about 37° C.), which is the normal human body temperature. Therefore, a boundary of the blood clot at 2.3 cm away from the centre of the blood clot centre will remain at the normal body temperature of 37° C. In view of the above, the treatment of the blood clot using the treatment transducer is safe.

In the following, a proof of concept experiment of the measurement transducer according to various embodiments, will be described.

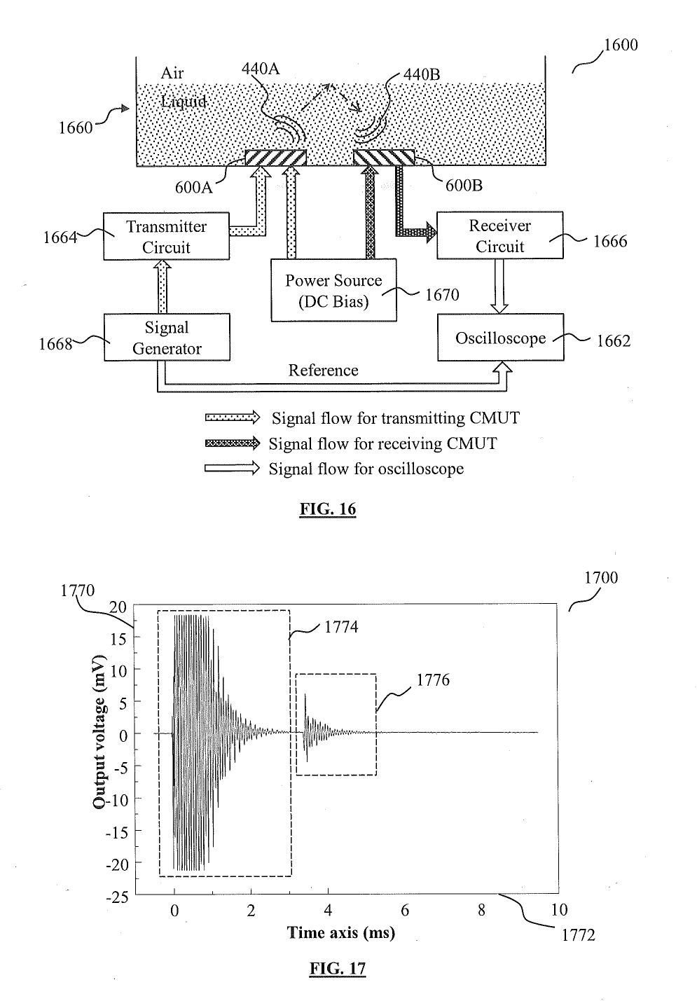

FIG. 16 shows an experiment set-up 1600, including a liquid bath 1660, a transmitting CMUT element 600A, a receiving CMUT element 600B, a transmitter circuit 1664, a receiver circuit 1666, a signal generator 1668, an oscilloscope 1662 and a power source 1670. The liquid bath 1660 contains a liquid which may be water or oil. The transmitting CMUT element 600A and the receiving CMUT element 600B may be both immersed into the liquid bath 1660, with a 20V DC bias supplied by the power source 1670 applied to a common bottom electrode of the transmitting CMUT element 600A and the receiving CMUT element 600B. The transmitting CMUT element may be first activated with a pulse signal (20V Vpp amplitude, 50 ns pulse width and 500 Hz repetition frequency) sent from the transmitter circuit 1664. The pulse signal may be generated by the signal generator 1668. The same signal may also be provided to the oscilloscope 1662 as a reference. The transmitting CMUT element 600A may then generate an outgoing acoustic wave 440A, which may be reflected at the liquid/air interface to become an incoming acoustic wave 440B which may be subsequently collected by the receiving CMUT element 600B. The receiving CMUT element 600B then provides an output current signal to the receiver circuit 1666 which includes a transimpedance amplifier (TIA). The receiver circuit 1666 may translate the output current signal to an amplified output voltage, which may then be captured by the oscilloscope 1662 at a sampling rate of 100 MHz.

For the proof of concept demonstration, the experiment was conducted with the experiment set-up 1600 with deionized (DI) water as the liquid in the liquid bath 1660, and then repeated with soybean oil as the liquid in the liquid bath 1660. The dynamic viscosities of the DI water and the soybean oil are is 1 cP and 80 cP, respectively.

FIG. 17 shows a graph 1700 which presents the variation of the output voltage in time, for an experiment conducted with the experiment set-up of FIG. 16, where DI water is used as the liquid in the liquid bath. Graph 1700 has a vertical axis 1770 and a horizontal axis 1772. The vertical axis 1770 indicates an output voltage while the horizontal axis indicates time. The graph 1700 includes a first part 1774 which shows an outgoing acoustic wave and a second part 1776 which shows an incoming acoustic wave. The incoming acoustic wave is a reflection of the outgoing acoustic wave from the air/DI water interface of the DI water bath.

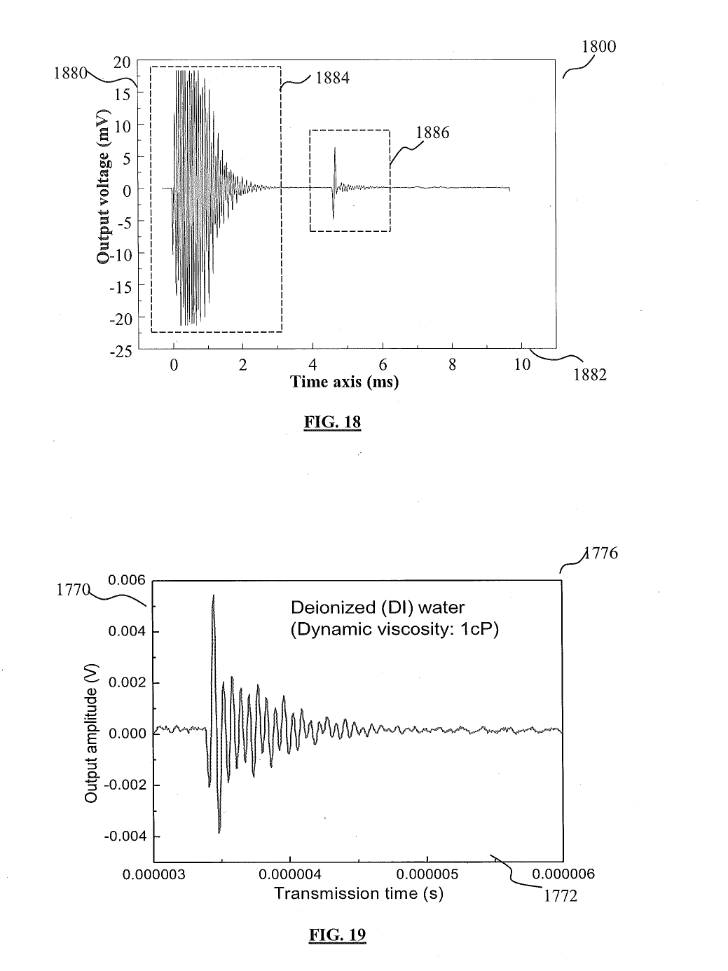

FIG. 18 shows a graph 1800 which presents the variation of the output voltage in time, for an experiment conducted with the experiment set-up of FIG. 16, where soybean oil is used as the liquid in the liquid bath. Graph 1800 has a vertical axis 1880 and a horizontal axis 1882. The vertical axis 1880 indicates an output voltage while the horizontal axis indicates time. The graph 1800 includes a first part 1884 which shows an outgoing acoustic wave and a second part 1886 which shows an incoming acoustic wave. The incoming acoustic wave is a reflection of the outgoing acoustic wave from the air/soybean oil interface of the oil bath.

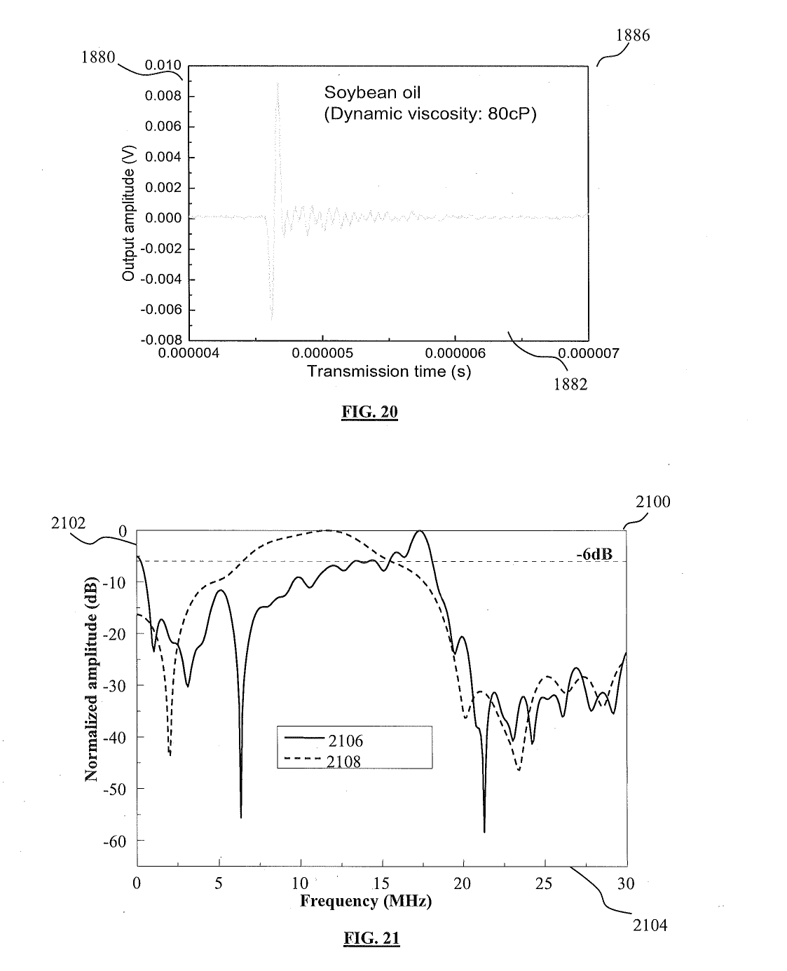

FIGS. 19 and 20 show amplified views of the second part 1776 of FIG. 17 and the second part 1886 of FIG. 18, respectively. The incoming acoustic wave of FIG. 19 demonstrates a distinct oscillation having a larger amplitude and a longer time duration as compared to the incoming acoustic wave of FIG. 20, as a result of the smaller damping effect in DI water, due to its lower dynamic viscosity (1 cP). The oscillation in the incoming acoustic wave of FIG. 20 is nearly negligible, due to the higher damping effect in soybean oil, as a result of a higher dynamic viscosity (80 cP).

FIG. 21 shows a graph 2100 having a vertical axis 2102 and a horizontal axis 2104. The vertical axis 2102 indicates a normalized amplitude while the horizontal axis 2104 indicates a frequency. The graph 2100 further includes a continuous line 2106 and a broken line 2108, representing the frequency responses of graph 1776 from FIG. 19 and graph 1886 from FIG. 20, respectively. Through performing Fourier transform to the time domain signals from graph 1776 of FIG. 19 and time domain signals from graph 1886 of FIG. 20, the frequency responses of the CMUT element in a DI water bath and in a soybean oil bath may be obtained. The continuous line 2106 shows that the center frequency and −6 dB bandwidth of the CMUT device are found to be 16.8 MHz and 2.65 MHz, respectively, in the DI water bath. The broken line 2108 shows that the center frequency will shift to a lower value of 11.1 MHz, and at the same time the −6 dB bandwidth increases to 9.12 MHz, when the CMUT device is operated in the soybean oil bath.

In view of the above, a viscosity value of the liquid in the liquid bath may be deduced from the centre frequency of the incoming acoustic wave, which is an echo of the outgoing acoustic wave. A measurement resolution of the viscosity may be dependent on the minimum frequency shift that is determinable. The minimum determinable frequency shift is inversely proportional to a sampling time of the oscilloscope. For example, given a sampling time of 1 ms, the frequency resolution will be 1 kHz. Considering the theoretical analysis results discussed above, the change gradient of the resonant frequency with respect to the viscosity is found to be around −72 kHz/cP when the viscosity falls around 80 cP. As a result, a 1 kHz frequency resolution may provide a viscosity measurement resolution as small as 0.014 cP ( 1/72).

While embodiments of the invention have been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced. It will be appreciated that common numerals, used in the relevant drawings, refer to components that serve a similar or the same purpose.

1. A thrombolysis device comprising:
a catheter configured to be inserted into a blood clot;
a treatment transducer coupled to the catheter, the treatment transducer configured to transmit acoustic waves;
a measurement transducer coupled to the catheter, the measurement transducer configured to transmit further acoustic waves and further configured to receive acoustic echoes, the acoustic echoes being reflections of the further acoustic waves from a boundary of the blood clot, wherein the measurement transducer is further configured to provide a measurement output;
a determination circuit configured to determine at least one of a viscosity of blood in a vicinity of the measurement transducer or a volume of the blood clot, based on the measurement output; and
a control circuit configured to generate control signals for controlling the treatment transducer, based on at least one from the group consisting of the viscosity of blood in the vicinity of the measurement transducer, the volume of the blood clot and a temperature measurement of the blood clot.

2. The thrombolysis device of claim 1, wherein the measurement output comprises at least one of a resonant frequency of the measurement transducer or a travelling time of the acoustic echoes.

3. The thrombolysis device of claim 2, wherein the viscosity of blood in the vicinity of the measurement transducer is determined based on the resonant frequency of the measurement transducer.

4. The thrombolysis device of claim 2, wherein the volume of the blood clot is determined based on the travelling time of the acoustic echoes.

5. The thrombolysis device of claim 1, wherein at least one of the treatment transducer or the measurement transducer is arranged on at least one of an end of the catheter or a sidewall of the catheter.

6. The thrombolysis device of claim 1, wherein the treatment transducer comprises a micromachined transducer element.

7. The thrombolysis device of claim 6, wherein the treatment transducer further comprises a front-end circuit configured to control the micromachined transducer element of the treatment transducer.

8. The thrombolysis device of claim 1, wherein the measurement transducer comprises a micromachined transducer element.

9. The thrombolysis device of claim 8, wherein the measurement transducer further comprises a front-end circuit configured to control the micromachined transducer element of the measurement transducer.

10. The thrombolysis device of claim 1, wherein the treatment transducer comprises a first micromachined transducer element and the measurement transducer comprises a second micromachined transducer element.

11. The thrombolysis device of claim 10, wherein the first micromachined transducer element and the second micromachined transducer element are fabricated on a common semiconductor device.

12. The thrombolysis device of claim 1, wherein the acoustic waves are continuous sound waves.

13. The thrombolysis device of claim 1, wherein the further acoustic waves are a series of pulses.

14. The thrombolysis device of claim 1, wherein the acoustic waves have a frequency within a range of 10 kHz to 100 kHz.

15. The thrombolysis device of claim 1, wherein the further acoustic waves have a frequency within a range of 1 MHz to 10 MHz.

16. The thrombolysis device of claim 1, wherein the control signals are configured to control at least one of a driving voltage of the treatment transducer or an operation duration of the treatment transducer.

17. The thrombolysis device of claim 1, wherein the determination circuit is further configured to determine the temperature measurement of the blood clot, based on the measurement output.

18. The thrombolysis device of claim 1, further comprising a temperature sensor coupled to the catheter, the temperature sensor configured to provide the temperature measurement of the blood clot.

19. The thrombolysis device of claim 18, wherein the temperature sensor is positioned within the catheter.

20. A method of operating a thrombolysis device, the method comprising:
inserting a catheter into a blood clot;
transmitting acoustic waves using a treatment transducer coupled to the catheter;
transmitting further acoustic waves and receiving acoustic echoes using a measurement transducer coupled to the catheter, the acoustic echoes being reflections of the further acoustic waves from a boundary of the blood clot;
providing a measurement output using the measurement transducer;
determining at least one of a viscosity of blood in a vicinity of the measurement transducer or a volume of the blood clot, based on the measurement output, using a determination circuit;
generating control signals for controlling the treatment transducer, based on at least one from the group consisting of the viscosity of blood in the vicinity of the measurement transducer, the volume of the blood clot and a temperature measurement of the blood clot, using a control circuit.
